SEQUENCE LISTING (3 sequences)

SEQ ID NO: 1 - DNA, 1261 bp, bacillus
gggagcttgctccctgatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagctagttggtgaggtaatggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggctgcaagaccgcaaggtttagccaatcccataaatctgttctcagttcggatcgcagtctgcaactcgactgcg

SEQ ID NO: 2 - DNA, 1398 bp, Bacillus
agtcgagcggacagatgggagcttgctccctgatgtcagcggcggacgggtgagtaacacgtgggtaacctgcctgtaagactgggataactccgggaaaccggggctaataccggatgcttgattgaaccgcatggttcaattataaaaggtggcttcggctatcacttaccgatggacccgcggcgccttaactaattggtgagggaacggctcaccaaggccacgatgcctaaccgacctgaaagggtgatcggccaccctgggactgaaacccggcccaaactcctacgggaggcagcagtaaggaatcttccgcaatggaccaaagtctgacggaacaaccccgcgtgagtgatgaagggtttcggatcgtaaaactctggtgttagggaagaaccagtaccgttcgaataaggcgggaccttgacggtacctaaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactgggaaacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagataggggttccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaagccgcgaggctaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacct

SEQ ID NO: 3 - DNA, 1071 bp, Pantoea
tgccgcgtgtatgaagaaggccttcgggttgtaaagtactttcagcggggaggaaggcgatgcggttaataaccgcgtcgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagcgaacttggcagagatgccttggtgccttcgggaacgctgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagtagggctacacacgtgctacaatggcgcatacaaagagaagcgacctcgcgagagcaagcggacctcacaaagtgcgtcgtagtccggatcggagtctgcaactcgactccgtgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttacca

(19) United States
(12) Patent Application Publication
(10) Pub. No.: US 2022/0287312 A1
(43) Pub. Date: Sep. 15, 2022

(54) BACTERIA AND METHODS OF USE THEREOF

(71) Applicant: DEAD SEA AND ARAVA SCIENCE CENTER, D.N. Dead Sea (IL)

(72) Inventors: Ashraf AL ASHHAB, East Jerusalem (IL); Yael BAR-LAVAN, Ein Yahav (IL); Gidon WINTERS, Moshav Idan (IL)

(21) Appl. No.: 17/634,980

(22) PCT Filed: Aug. 12, 2020
(86) PCT No.: PCT/IL2020/050890

§ 371 Date: Feb. 13, 2022

Related U.S. Application Data
(60) Provisional application No. 62/885,386, filed on Aug. 12, 2019.

(51) Int. Cl. A01N 63/22; C12N 1/20

(52) U.S. Cl. CPC A01N 63/22; C12N 1/205; C12R 2001/125

(57) ABSTRACT

The present invention is directed to bacteria, compositions comprising the bacteria and methods of use thereof, including methods for inhibiting fungus growth on plants.

### CROSS REFERENCE TO RELATED APPLICATIONS

This application claims the benefit of priority of U.S. Provisional Patent Application No. 62/885,386, filed Aug. 12, 2019, the contents of which are all incorporated herein by reference in their entirety.

### FIELD OF INVENTION

The present invention, in some embodiments thereof, is in the field of bacterial cells, and methods of use thereof, including for fungus growth inhibition.

### BACKGROUND OF THE INVENTION

Microbial populations of plant surfaces play an important role in the wellbeing of their hosts. These microbes, referred to as “plant probiotic bacteria”, enhance the plant's ability to acquire nutrients, contribute to pathogen and predator resistance, improve resistance to environmental stresses (e.g., tolerance to heat, cold, drought, salinity, etc.), modulate plant immunity and development.

Diseases caused by phytopathogenic fungi are considered extremely harmful and are widespread worldwide. Fungal growth on plants can inhibit production of foliage, fruit, or seeds, as well as reduce the quality and quantity of the harvested crop. At present, the control of these diseases is based fundamentally on the use of chemical fungicides, despite their high toxicity and their low biodegradability. The use of such chemical fungicides is mainly due to the lack of effective and environmentally safer alternatives. Therefore, the use of probiotic bacteria can provide an effective approach for preventing fungal infection in plants without significant adverse side effects to the plant or to the environment.

Antifungal activity has significance in four areas: development of therapeutic, antifungal drugs, development of plant protection agents, suppression of fungal colonisation/proliferation within the fungus carrying organism resulting in modification of the pattern of certain fungus carrying organism infections, and reduction in the efficiency of isolation of fungal pathogens from specimens.

A variety of mechanisms have been put forward to explain growth inhibition of one organism by another. For example, competition for a limited supply of nutrients, production of siderophores, antibiotics, enzymes, and volatile substances.

Currently, though several “plant probiotic bacteria” have been developed as microbe-based plant biofertilizers and biopesticides, while the list still long—only a small portion of microbes are culturable. Many microbes thriving under extreme conditions, such as the desert, may hold a significant potential for many agricultural and biotechnological applications.

Therefore, new “plant probiotic bacteria” applicable to agriculture in arid environments are greatly needed.

### SUMMARY OF THE INVENTION

According to some embodiments, the invention is directed to bacteria having antifungal activity against phytopathogenic fungal species that have significant impact on many agricultural crops.

According to one aspect, there is provided isolated bacteria comprising a polynucleotide sequence of SEQ ID NO: 1.

According to another aspect, there is provided isolated bacteria comprising a polynucleotide sequence of SEQ ID NO: 2.

According to another aspect, there is provided a composition comprising bacteria, wherein at least 90% of the bacteria are bacteria comprising a polynucleotide sequence of SEQ ID NO: 1, bacteria comprising a polynucleotide sequence of SEQ ID NO: 2, or both, and an artificial carrier or diluent.

According to another aspect, there is provided a method for inhibiting a fungus, comprising contacting the fungus with any one of: (a) isolated bacteria comprising a polynucleotide sequence of SEQ ID NO: 1; (b) isolated bacteria comprising a polynucleotide sequence of SEQ ID NO: 2; (c) isolated bacteria comprising a polynucleotide sequence of SEQ ID NO: 3; (d) any combination of (a), (b), and (c); and (e) an effective amount of any one of the herein disclosed composition, or a composition comprising the isolated bacteria comprising a polynucleotide of SEQ ID NO: 3, thereby inhibiting the fungus.

In some embodiment, the isolated bacteria, belongs to Bacillus.

In some embodiment, the isolated bacteria has a fungicidal activity.

In some embodiment, the composition, further comprises bacteria comprising a polynucleotide sequence of SEQ ID NO: 3.

In some embodiment, the bacteria comprising a polynucleotide sequence of SEQ ID NO: 3, belongs to Pantoea.

In some embodiment, the polynucleotide sequence is a 16S-rDNA sequence.

In some embodiment, the fungus is selected from the group consisting of: Pythium, Botrytis, Rhizoctonia, and Fusarium.

In some embodiment, contacting is contacting on a plant surface.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1B: A bar graph (1A) and a photograph (1B) showing bacterial antifungal activity. 1A: Growth inhibition of Fusarium with 6 different bacteria from Acacia leaves was quantified. 1B: Qualitative growth inhibition of the fungus is shown for 4 of these 6 bacteria. Fungal growth inhibition was recorded 72 hours after treatment with the bacteria disclosed herein (top row), and after one week of incubation with the bacteria (bottom row).

FIGS. 2A-2B: A bar graph (2A) and a photograph (2B) showing bacterial antifungal activity. 2A: Growth inhibition of Botrytis with 6 different bacteria from Acacia leaves was quantified. 2B: Qualitative growth inhibition of the fungus is shown for 4 of these 6 bacteria. Fungal growth inhibition was recorded 72 hours after treatment with the bacteria disclosed herein (top row), and after one week of incubation with the bacteria (bottom row).

FIGS. 3A-3F: Photographs showing that bacteria protect leaves of pepper from Botrytis cinerea: (3A) fungus-alone treatment; (3B) isolate 25.5; (3C) isolate 284.2.8; (3D) isolate 284.2.9; (3E) isolate 284.2.5; and (3F) isolate 284.2.4.

FIGS. 4A-4I: Photographs showing the biocontrol activity of isolates 25.5 and 284.2.9 on infected pepper plants. (4A-4C) fungus-alone treatment; (4D-4E) isolate 25.5; (4F-4G) isolate 284.2.9; and (4H-4I) control (treatment where no bacteria or fungal infection applied).

FIGS. 5A-5B: Photographs showing the biocontrol activity of a commercially-available bio-based formulation (5A) compared to isolate 209.2 (5B).

FIG. 6: A scheme of a phylogenetic tree comprising contigs: 3, 4 and 7 (encircled).

FIG. 7: A scheme of a phylogenetic tree comprising contigs: 1 and 10 (encircled).

FIG. 8: A scheme of a phylogenetic tree comprising contig 5 (encircled).

### DETAILED DESCRIPTION OF THE INVENTION

The present invention, in some embodiments, provides bacteria, compositions comprising the bacteria and methods of use thereof.

According to one embodiment, the present invention provides bacteria comprising the polynucleotide sequence:

(SEQ ID NO: 1)
GGGAGCTTGCTCCCTGATGTTAGCGGCGGACGGGTGAGTAACACGTGGGT
AACCTGCCTGTAAGACTGGGATAACTCCGGGAAACCGGGGCTAATACCGG
ATGCTTGTTTGAACCGCATGGTTCAAACATAAAAGGTGGCTTCGGCTACC
ACTTACAGATGGACCCGCGGCGCATTAGCTAGTTGGTGAGGTAATGGCTC
ACCAAGGCAACGATGCGTAGCCGACCTGAGAGGCTTGATCGGCCACACTG
GGACTGAGACACGGCCCAGACTCCTACGGGAGGCAGCAGTAGGGAATCTT
CCGCAATGGACGAAAGTCTGACGGAGCAACGCCGCGTGAGTGATGAAGGT
TTTCGGATCGTAAAGCTCTGTTGTTAGGGAAGAACAAGTACCGTTCGAAT
AGGGCGGTACCTTGACGGTACCTAACCAGAAAGCCACGGCTAACTACGTG
CCAGCAGCCGCGGTAATACGTAGGTGGCAAGCGTTGTCCGGAATTATTGG
GCGTAAAGGGCTCGCAGGCGGTTTCTTAAGTCTGATGTGAAAGCCCCCGG
CTCAACCGGGGAGGGTCATTGGAAACTGGGGAACTTGAGTGCAGAAGAGG
AGAGTGGAATTCCACGTGTAGCGGTGAAATGCGTAGAGATGTGGAGGAAC
ACCAGTGGCGAAGGCGACTCTCTGGTCTGTAACTGACGCTGAGGAGCGAA
AGCGTGGGGAGCGAACAGGATTAGATACCCTGGTAGTCCACGCCGTAAAC
GATGAGTGCTAAGTGTTAGGGGGTTTCCGCCCCTTAGTGCTGCAGCTAAC
GCATTAAGCACTCCGCCTGGGGAGTACGGTCGCAAGACTGAAACTCAAAG
GAATTGACGGGGGCCCGCACAAGCGGTGGAGCATGTGGTTTAATTCGAAG
CAACGCGAAGAACCTTACCAGGTCTTGACATCCTCTGACAACCCTAGAGA
TAGGGCTTTCCCTTCGGGGACAGAGTGACAGGTGGTGCATGGTTGTCGTC
AGCTCGTGTCGTGAGATGTTGGGTTAAGTCCCGCAACGAGCGCAACCCTT
GATCTTAGTTGCCAGCATTCAGTTGGGCACTCTAAGGTGACTGCCGGTGA
CAAACCGGAGGAAGGTGGGGATGACGTCAAATCATCATGCCCCTTATGAC
CTGGGCTACACACGTGCTACAATGGACAGAACAAAGGGCTGCAAGACCGC
AAGGTTTAGCCAATCCCATAAATCTGTTCTCAGTTCGGATCGCAGTCTGC
AACTCGACTGCG.

According to some embodiments, the bacteria comprises a polynucleotide sequence comprising 1 to 5, 1 to 4, 1 to 3, 2 to 4, or 3 to 5 substitutions compared to SEQ ID NO: 1. Each possibility represents a separate embodiment of the invention. According to some embodiments, the bacteria comprises a polynucleotide sequence comprising at most 5, at most 4, at most 3, at most 2, or at most 1 substitution compared to SEQ ID NO: 1, or any value and range therebetween. Each possibility represents a separate embodiment of the invention.

According to one embodiment, the present invention provides bacteria comprising the polynucleotide sequence:

(SEQ ID NO: 2)
AGTCGAGCGGACAGATGGGAGCTTGCTCCCTGATGTCAGCGGCGGACGGG
TGAGTAACACGTGGGTAACCTGCCTGTAAGACTGGGATAACTCCGGGAAA
CCGGGGCTAATACCGGATGCTTGATTGAACCGCATGGTTCAATTATAAAA
GGTGGCTTCGGCTATCACTTACCGATGGACCCGCGGCGCCTTAACTAATT
GGTGAGGGAACGGCTCACCAAGGCCACGATGCCTAACCGACCTGAAAGGG
TGATCGGCCACCCTGGGACTGAAACCCGGCCCAAACTCCTACGGGAGGCA
GCAGTAAGGAATCTTCCGCAATGGACCAAAGTCTGACGGAACAACCCCGC
GTGAGTGATGAAGGGTTTCGGATCGTAAAACTCTGGTGTTAGGGAAGAAC
CAGTACCGTTCGAATAAGGCGGGACCTTGACGGTACCTAACCAGAAAGCC
ACGGCTAACTACGTGCCAGCAGCCGCGGTAATACGTAGGTGGCAAGCGTT
GTCCGGAATTATTGGGCGTAAAGCGCGCGCAGGCGGTTTCTTAAGTCTGA
TGTGAAAGCCCCCGGCTCAACCGGGGAGGGTCATTGGAAACTGGGAAACT
TGAGTGCAGAAGAGGAGAGTGGAATTCCACGTGTAGCGGTGAAATGCGTA
GAGATGTGGAGGAACACCAGTGGCGAAGGCGACTCTCTGGTCTGTAACTG
ACGCTGAGGCGCGAAAGCGTGGGGAGCGAACAGGATTAGATACCCTGGTA
GTCCACGCCGTAAACGATGAGTGCTAAGTGTTAGAGGGTTTCCGCCCTTT
AGTGCTGCAGCAAACGCATTAAGCACTCCGCCTGGGGAGTACGGTCGCAA
GACTGAAACTCAAAGGAATTGACGGGGGCCCGCACAAGCGGTGGAGCATG
TGGTTTAATTCGAAGCAACGCGAAGAACCTTACCAGGTCTTGACATCCTC
TGACAACCCTAGAGATAGGGGTTCCCCTTCGGGGGCAGAGTGACAGGTGG
TGCATGGTTGTCGTCAGCTCGTGTCGTGAGATGTTGGGTTAAGTCCCGCA
ACGAGCGCAACCCTTGATCTTAGTTGCCAGCATTCAGTTGGGCACTCTAA
GGTGACTGCCGGTGACAAACCGGAGGAAGGTGGGGATGACGTCAAATCAT
CATGCCCCTTATGACCTGGGCTACACACGTGCTACAATGGGCAGAACAAA
GGGCAGCGAAGCCGCGAGGCTAAGCCAATCCCACAAATCTGTTCTCAGTT
CGGATCGCAGTCTGCAACTCGACTGCGTGAAGCTGGAATCGCTAGTAATC
GCGGATCAGCATGCCGCGGTGAATACGTTCCCGGGCCTTGTACACACCGC
CCGTCACACCACGAGAGTTTGTAACACCCGAAGTCGGTGAGGTAACCT.

According to some embodiments, the bacteria comprises a polynucleotide sequence comprising 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, substitutions compared to SEQ ID NO: 2. Each possibility represents a separate embodiment of the invention. According to some embodiments, the bacteria comprises a polynucleotide sequence comprising at most 22, at most 20, at most 18, at most 15, at most 12, at most 9, at most 6, at most 3, or at most 1 substitution compared to SEQ ID NO: 2. Each possibility represents a separate embodiment of the present invention.

In one embodiment, a bacteria as described herein is an isolated bacteria. In one embodiment, the bacteria is in a composition comprising an artificial carrier.

In some embodiments, the bacteria of the invention is isolated from a plant. According to some embodiments, bacteria of the invention is isolated from a plant part selected from: seed, leaf, root, shoot, stem, flower, fruit, stolon, bulb, tuber, corm, bud, or any combination thereof. In some embodiments, the bacteria of the invention is isolated from a growth media comprising plant material, for example, a soil sample. Methods for isolating bacteria are common, such as exemplified hereinbelow, and would be apparent to one of ordinary skill in the art.

According to some embodiments, the bacteria have fungicidal activity.

As used herein, the term “fungicidal activity” refers to the ability to inhibit fungal activity, inhibit fungal growth, inhibit fungal spread, eliminate fungi, or any combination thereof. In some embodiments, the terms “inhibit”, “treat”, and “prevent” are interchangeable.

In some embodiments, inhibiting is reducing the growth rate of a fungus. In some embodiments, inhibiting is killing a fungus. In some embodiments, inhibiting is blocking or reducing fungal DNA replication and/or cell division of a fungus. In some embodiments, inhibiting is blocking or reducing fungal protein production or secretion from the fungus. In some embodiments, inhibiting is reducing fungal spores' production and/or germination and/or dissemination thereof. In some embodiments, inhibiting is reducing fungal toxin production or secretion thereof.

In some embodiments, inhibiting is a reduction of at least 5%, at least 10%, at least 20%, at least 35%, at least 40%, at least 50%, at least 70%, at least 75%, at least 80%, at least 85%, at least 90%, at least 95%, or at least 99%, or any range and value therebetween, compared to a control. Each possibility represents a separate embodiment of the invention. In some embodiments, inhibiting is a reduction of 5-25%, 15-35%, 20-45%, 30-65%, 50-100%, 50-90%, 50-80%, 60-90%, 60-80%, 70-90%, 80-100%, 80-95%, 80-90%, or 90-100%, compared to a control. Each possibility represents a separate embodiment of the invention.

As used herein, the term “control”, refers to a fungus that was not contacted with the bacteria as discussed herein. In some embodiments, a control fungus is a fungus that is contacted with a fungicide excluding any one of the bacteria disclosed herein, or any composition comprising thereof, for example, a commercially-available fungicide.

Methods for determining fungicidal activity are common and would be apparent to one of ordinary skill in the art.

In some embodiments, a fungus is selected from the genera: Pythium, Botrytis, Rhizoctonia, or Fusarium.

According to some embodiments, the present invention is directed to a composition comprising the bacteria disclosed herein. In some embodiments, at least 70%, at least 75%, at least 80%, at least 85%, at least 90%, at least 95%, at least 97%, at least 99%, or 100% of the bacteria in the composition is the bacteria disclosed herein, or any value and range therebetween. Each possibility represents a separate embodiment of the invention. According to some embodiments, 70-100%, 75-95%, 70-90%, 80-90%, or 90-100%, of the bacteria in the composition is the bacteria disclosed herein. Each possibility represents a separate embodiment of the invention.

In some embodiments, at least 30%, at least 40%, at least 50%, at least 60%, at least 70%, at least 80%, at least 90%, at least 95%, at least 99%, or 100% of the bacteria in the composition comprise the polynucleotide sequence of SEQ ID NO: 1, or any value and range therebetween. Each possibility represents a separate embodiment of the invention. In some embodiments, 30-100%, 40-90%, 40-80%, 40-70%, 40-60%, 60-90%, 70-90%, 70-100%, 80-100%, or 90-100%, of the bacteria of the composition comprise the polynucleotide sequence of SEQ ID NO: 1. Each possibility represents a separate embodiment of the invention. In some embodiments, at least 30%, at least 40%, at least 50%, at least 60%, at least 70%, at least 80%, at least 90%, at least 95%, at least 99%, or 100% of the bacteria of the composition comprise the polynucleotide sequence of SEQ ID NO: 2, or any value and range therebetween. Each possibility represents a separate embodiment of the present invention. In some embodiments, 30-100%, 40-90%, 40-80%, 40-70%, 40-60%, 60-90%, 70-90%, 70-100%, 80-100%, 90-100%, of the bacteria of the composition comprise the polynucleotide sequence of SEQ ID NO: 2. Each possibility represents a separate embodiment of the present invention.

In some embodiments, the composition comprises bacteria comprising the polynucleotide sequence of SEQ ID NO: 1 and bacteria comprising the polynucleotide sequence of SEQ ID NO: 2.

In some embodiments, bacteria comprising the polynucleotide sequence of SEQ ID NO: 1 and bacteria comprising the polynucleotide sequence of SEQ ID NO: 2 are present in the composition in an amount ranging from 20:1 (CFU/CFU) to 1:20 (CFU/CFU).

In some embodiments, a ratio ranging from 20:1 (CFU/CFU) to 1:20 (CFU/CFU) comprises 15:1 (CFU/CFU) to 1:20 (CFU/CFU), 10:1 (CFU/CFU) to 1:20 (CFU/CFU), 8:1 (CFU/CFU) to 1:20 (CFU/CFU), 5:1 (CFU/CFU) to 1:20 (CFU/CFU), 20:1 (CFU/CFU) to 1:15 (CFU/CFU), 20:1 (CFU/CFU) to 1:10 (CFU/CFU), 20:1 (CFU/CFU) to 1:5 (CFU/CFU), 15:1 (CFU/CFU) to 1:10 (CFU/CFU), 10:1 (CFU/CFU) to 1:10 (CFU/CFU), 5:1 (CFU/CFU) to 1:10 (CFU/CFU), 20:1 (CFU/CFU) to 1:2 (CFU/CFU), or 2:1 (CFU/CFU) to 1:5 (CFU/CFU).

As used herein, the term “CFU” refers to colony forming unit.

In some embodiments, the composition comprises the bacteria disclosed herein and an acceptable carrier or diluent. As used herein, the term “carrier” refers to a diluent, adjuvant, excipient, or vehicle with which the active compound is administered.

In some embodiments, the carrier is an agriculturally-acceptable carrier. Agricultural carriers may be soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay.

The carrier may comprise, in total, from about 0.1% to about 99.99999% by weight of the compositions presented herein.

According to some embodiments, the composition is a liquid, a solid, or an aerosol.

According to some embodiments, the present invention is directed to a composition for use in fungus inhibition, wherein the composition comprises bacteria comprising a polynucleotide sequence of SEQ ID NO: 1, bacteria comprising a polynucleotide sequence of SEQ ID NO: 2, bacteria comprising a polynucleotide sequence of SEQ ID NO: 3, or any combination thereof. According to some embodiments, the present invention is directed to a composition for use in fungus inhibition, wherein the bacteria content of the composition comprises at least 80%: bacteria comprising a polynucleotide sequence of SEQ ID NO: 1, bacteria comprising a polynucleotide sequence of SEQ ID NO: 2, bacteria comprising a polynucleotide sequence of SEQ ID NO: 3, or any combination thereof. According to some embodiments, the present invention is directed to a composition for use in fungus inhibition, wherein the bacteria content of the composition comprises at least 85%: bacteria comprising a polynucleotide sequence of SEQ ID NO: 1, bacteria comprising a polynucleotide sequence of SEQ ID NO: 2, bacteria comprising a polynucleotide sequence of SEQ ID NO: 3, or any combination thereof. According to some embodiments, the present invention is directed to a composition for use in fungus inhibition, wherein the bacteria content of the composition comprises at least 90%: bacteria comprising a polynucleotide sequence of SEQ ID NO: 1, bacteria comprising a polynucleotide sequence of SEQ ID NO: 2, bacteria comprising a polynucleotide sequence of SEQ ID NO: 3, or any combination thereof. According to some embodiments, the present invention is directed to a composition for use in fungus inhibition, wherein the bacteria content of the composition comprises at least 95%: bacteria comprising a polynucleotide sequence of SEQ ID NO: 1, bacteria comprising a polynucleotide sequence of SEQ ID NO: 2, bacteria comprising a polynucleotide sequence of SEQ ID NO: 3, or any combination thereof. According to some embodiments, the present invention is directed to a composition for use in fungus inhibition, wherein the bacteria content of the composition comprises at least 99%: bacteria comprising a polynucleotide sequence of SEQ ID NO: 1, bacteria comprising a polynucleotide sequence of SEQ ID NO: 2, bacteria comprising a polynucleotide sequence of SEQ ID NO: 3, or any combination thereof.

In some embodiments, SEQ ID NO: 3 comprises the polynucleotide sequence:

TGCCGCGTGTATGAAGAAGGCCTTCGGGTTGTAAAGTACTTTCAGCGGGG
AGGAAGGCGATGCGGTTAATAACCGCGTCGATTGACGTTACCCGCAGAAG
AAGCACCGGCTAACTCCGTGCCAGCAGCCGCGGTAATACGGAGGGTGCAA
GCGTTAATCGGAATTACTGGGCGTAAAGCGCACGCAGGCGGTCTGTTAAG
TCAGATGTGAAATCCCCGGGCTTAACCTGGGAACTGCATTTGAAACTGGC
AGGCTTGAGTCTCGTAGAGGGGGGTAGAATTCCAGGTGTAGCGGTGAAAT
GCGTAGAGATCTGGAGGAATACCGGTGGCGAAGGCGGCCCCCTGGACGAA
GACTGACGCTCAGGTGCGAAAGCGTGGGGAGCAAACAGGATTAGATACCC
TGGTAGTCCACGCCGTAAACGATGTCGACTTGGAGGTTGTTCCCTTGAGG
AGTGGCTTCCGGAGCTAACGCGTTAAGTCGACCGCCTGGGGAGTACGGCC
GCAAGGTTAAAACTCAAATGAATTGACGGGGGCCCGCACAAGCGGTGGAG
CATGTGGTTTAATTCGATGCAACGCGAAGAACCTTACCTACTCTTGACAT
CCAGCGAACTTGGCAGAGATGCCTTGGTGCCTTCGGGAACGCTGAGACAG
GTGCTGCATGGCTGTCGTCAGCTCGTGTTGTGAAATGTTGGGTTAAGTCC
CGCAACGAGCGCAACCCTTATCCTTTGTTGCCAGCGATTCGGTCGGGAAC
TCAAAGGAGACTGCCGGTGATAAACCGGAGGAAGGTGGGGATGACGTCAA
GTCATCATGGCCCTTACGAGTAGGGCTACACACGTGCTACAATGGCGCAT
ACAAAGAGAAGCGACCTCGCGAGAGCAAGCGGACCTCACAAAGTGCGTCG
TAGTCCGGATCGGAGTCTGCAACTCGACTCCGTGAAGTCGGAATCGCTAG
TAATCGTGGATCAGAATGCCACGGTGAATACGTTCCCGGGCCTTGTACAC
ACCGCCCGTCACACCATGGGAGTGGGTTGCAAAAGAAGTAGGTAGCTTAA
CCTTCGGGAGGGCGCTTACCA.

In some embodiments, the composition for use in fungus inhibition comprises the bacteria comprising the polynucleotide sequence of SEQ ID NO: 1 and the bacteria comprising the polynucleotide sequence of SEQ ID NO: 2.

In some embodiments, the composition for use in fungus inhibition comprises the bacteria comprising the polynucleotide sequence of SEQ ID NO: 1 and the bacteria comprising the polynucleotide sequence of SEQ ID NO: 3.

In some embodiments, the composition for use in fungus inhibition comprises the bacteria comprising the polynucleotide sequence of SEQ ID NO: 2 and the bacteria comprising the polynucleotide sequence of SEQ ID NO: 3.

In some embodiments, the composition for use in fungus inhibition comprises the bacteria comprising the polynucleotide sequence of SEQ ID NO: 1, the bacteria comprising the polynucleotide sequence of SEQ ID NO: 2, and the bacteria comprising the polynucleotide sequence of SEQ ID NO: 3.

According to some embodiments, the composition for use in fungus inhibition comprises at least 10%, at least 20%, at least 30%, at least 40%, at least 50%, at least 60%, at least 70%, at least 80%, at least 90%, at least 95%, or at least 99%, of the bacteria comprising the polynucleotide sequence of SEQ ID NO: 1, or any value and range therebetween. Each possibility represents a separate embodiment of the invention. In some embodiments, 10-30%, 20-50%, 30-100%, 40-90%, 40-80%, 40-70%, 40-60%, 60-90%, 70-90%, 70-100%, 80-100%, or 90-100%, of the bacteria in the composition comprise the polynucleotide sequence of SEQ ID NO: 1. Each possibility represents a separate embodiment of the invention. In some embodiments, at least 10%, at least 20%, at least 30%, at least at least 40%, at least 50%, at least 60%, at least 70%, at least 80%, at least 90%, at least 95%, or at least 99%, of the bacteria in the composition comprise the polynucleotide sequence of SEQ ID NO: 2, or any value and range therebetween. Each possibility represents a separate embodiment of the invention. In some embodiments, 10-30%, 20-50%, 30-100%, 40-90%, 40-80%, 40-70%, 40-60%, 60-90%, 70-90%, 70-100%, 80-100%, or 90-100%, of the bacteria in the composition comprise the polynucleotide sequence of SEQ ID NO: 2. Each possibility represents a separate embodiment of the invention. In some embodiments, at least 10%, at least 20%, at least 30%, at least 40%, at least 50%, at least 60%, at least 70%, at least 80%, at least 90%, at least 95%, or at least 99%, of the bacteria in the composition comprise the polynucleotide sequence of SEQ ID NO: 3, or any value and range therebetween. Each possibility represents a separate embodiment of the invention. In some embodiments, 10-30%, 20-50%, 30-100%, 40-90%, 40-80%, 40-70%, 40-60%, 60-90%, 70-90%, 70-100%, 80-100%, or 90-100%, of the bacteria in the composition comprise the polynucleotide sequence of SEQ ID NO: 3. Each possibility represents a separate embodiment of the present invention.

In some embodiments, the composition further comprises an antifungal agent. As used herein, the term “antifungal agent” encompasses any agent capable of inhibiting a fungus, as described herein. In some embodiments, the antifungal agent comprises bacteria, fungi, or a chemical fungicide. According to some embodiments, the composition further comprises Bacillus pumilus, Bacillus atrophaeus, Bacillus subtilis, or any combination thereof.

In some embodiments, the bacteria disclosed herein belongs to the genera Bacillus or Pantoea.

In some embodiments, the polynucleotide sequence is a 16S-ribosomal DNA (16S-rDNA) sequence. In some embodiments, the polynucleotide sequence is a fragment of the 16S-rDNA.

The terms “16S-ribosomal DNA” and “16S rDNA” are used herein interchangeably, and refer to the gene encoding the bacterial 16S ribosomal RNA.

### Method

According to some embodiments, the present invention is directed to a method for inhibiting a fungus comprising, contacting the fungus with the bacteria disclosed herein, or with an effective amount of a composition comprising thereof.

According to some embodiments, “an effective amount” refers to an amount useful for reducing the severity of a disease, or damage, reduce the severity of symptoms associated therewith, or provide improvement to a plant's wellbeing.

As used herein, the term “contacting” encompasses depositing, applying, administering, providing, touching, and the like, which are used herein interchangeably.

In one embodiment, inhibiting fungus comprises inhibiting fungi.

According to some embodiments, a composition applied to a surface is contacting the fungus. In some embodiments, the fungus resides, adheres, is attached, is bound, or any combination thereof, to the surface.

As used herein, the term “surface” encompasses any area comprising the fungus which the bacteria disclosed herein, or a composition comprising thereof, can be applied thereto. In some embodiments, the surface is a natural surface. In some embodiments, the surface is a man-made surface, a synthetic surface, or an artificial surface.

According to some embodiments, the surface is a surface of a plant or a plant part. According to some embodiments, the surface is soil comprising a plant or a plant part.

In some embodiments, the method is for protecting a plant from a fungus. As used herein, protecting a plant from a fungus comprises treating a plant infected with a fungus, preventing fungus infection in a plant, or both. In some embodiments, bacteria or a mixture of bacteria as described herein inhibits the growth of fungus. In some embodiments, bacteria or a mixture of bacteria as described herein inhibits the proliferation of fungus. In some embodiments, bacteria or a mixture of bacteria as described herein eliminates fungus. In some embodiments, bacteria or a mixture of bacteria as described herein applied to a plant or surface thereof, do not harm the plant.

In one embodiment, “a surface susceptible to fungus growth” includes a plant or an animal surface. In one embodiment, “a surface susceptible to fungus growth” comprises a topical surface. In one embodiment, “a surface susceptible to fungus growth” comprises a plant leaf. In one embodiment, “a surface susceptible to fungus growth” comprises a plant root. In one embodiment, “a surface susceptible to fungus growth” comprises a plant stem. In one embodiment, “a surface susceptible to fungus growth” comprises a tree trunk. In one embodiment, “a surface susceptible to fungus growth” comprises a tree branch. In one embodiment, “a surface susceptible to fungus growth” comprises a tree crown. In one embodiment, “a surface susceptible to fungus growth” comprises a fruit. In one embodiment, “a surface susceptible to fungus growth” comprises a vegetable. In one embodiment, “a surface susceptible to fungus growth” comprises a flower.

In some embodiments, “treating” encompasses alleviation, reduction in the severity, or inhibition, of at least one symptom related to fungi plant infection. According to some embodiments, the plant symptoms include growth inhibition, abnormal growth, necrosis, leaf spots, blight, scab, rots, damping-off, warts, chlorosis, and leaf curls.

In some embodiments, the method is for preventing a phytopathogenic fungus from any one of: settling on a plant, infesting the plant, inducing decay of the plant or a part thereof, or any combination thereof. In some embodiments, the method is for treating a fungus infestation or contamination. In some embodiments, the method is a prophylactic method.

According to some embodiments, the composition is applied to a plant or a part thereof, infected by a fungus. According to some embodiments, the composition is applied to a plant uninfected by a fungus as means of preventing fungus growth. According to some embodiments, the composition is applied to a plant uninfected by a fungus as means of preventing fungus spread on the plant.

According to some embodiments, the composition can be applied by means of spraying, sprinkling, watering, immersing, drip irrigation, or any equivalent thereof, or any combination thereof. According to some embodiments, the composition can be applied by any means known to one of skill in the art.

In one embodiment, the plant is an angiosperm. In one embodiment, the plant is selected from: Acacia, Atriplex, Tamarix, Cucumis, Capsicum, Solanum, Rheum, Galanthus, Limnanthes, and Fragaria. In one embodiment, the plant is selected from: Acacia raddiana, Acacia tortilis, Atriplex halimus, Tamarix aphylla, Cucumis melo, Capsicum annuum.

As used herein, the term “about” when combined with a value refers to plus and minus 10% of the reference value. For example, a length of about 1,000 nanometers (nm) refers to a length of 1,000±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the bacteria” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Example 1

### Antifungal Activity of the Bacteria of the Invention Against Fusarium solani and Botrytis cinerea

In search of “probiotic bacteria”, having antifungal activity, bacteria from arid environment were isolated and examined. Leaf samples were collected from trees at different locations of the arid Arava region of Israel. Leaf samples were collected from both Acacia raddiana and Acacia tortilis, on December 2016 and March 2017. In addition, leaf samples were also collected from Atriplex halimus and Tamarix aphylla in March 2018 (Table 1). At each sampling campaign, 5-10 g of leaves were collected and inserted into 15 ml sterile tubes and placed on ice. Upon reaching the laboratory (within 2 hours), 2 g of each leaf sample were placed in 1 ml of PBS solution and subjected to vortex at maximum speed for 10 minutes. Then the supernatant was mixed with 30% glycerol and placed in −80° C. for further experiments.

TABLE 1
Leaves sample collection of the different sampling campaigns.

| Campaign | collection date | Location |
|---|---|---|
| First | Dec. 18, 2016 | Shizaf |
| Second | Mar. 10, 2017 | Shizaf |
| Third | Mar. 7, 2018 | Dead Sea |

100 μl of the leaves' suspension were inoculated on LB agar for three days at 37° C. Upon first step of incubation, different bacterial colonies were picked up and plated on LB medium to ensure collecting pure isolates (Table 2). Each isolate was then grown on LB media and collected by adding 1 ml of PBS to the agar plate and scratch to 2 ml sterile tube. Then 30% glycerol (v/v) was added to each tube and placed in −80° C. for further experiments.

TABLE 2
Number of obtained isolates collected from different plant leaves
in each sampling campaign.

| Collection date | Number of isolates |
|---|---|
| First | 12 |
| Second | 33 |
| Third | 48 |

All isolates were tested for their antifungal properties, against Fusarium solani, (isolated and cultured from Melon plants) and Botrytis cinerea. All tests were carried out on 0.25% potato dextrose agar (PDA) growth medium where a plague of fungus was placed in the middle of the petri dish. The bacterial isolates were placed in the four corners surrounding the fungal plague and incubated for 7 days at 20° C. The petri dishes were tested every day using Image J software to examine the fungus growth. The fungal growth inhibition was recorded after 72 hours, and after one week of incubation.

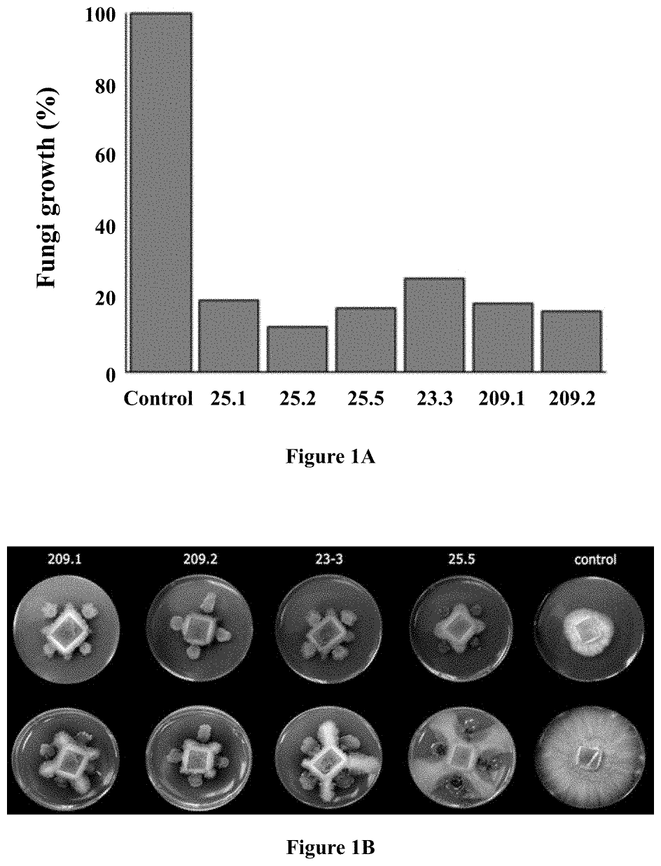

Out of all the tested isolates (Table 2), six isolates, namely 25.1, 25.2, 25.5, 23.3, 209.1, and 209.2, showed significant antifungal properties against Fusarium solani (FIG. 1). Specifically, isolates 25.2, 209.2, 209.1, and 25.5 exhibited the most effective growth inhibition against Fusarium solani of 87.6%, 83.1%, 82.3%, and 80.9%, respectively.

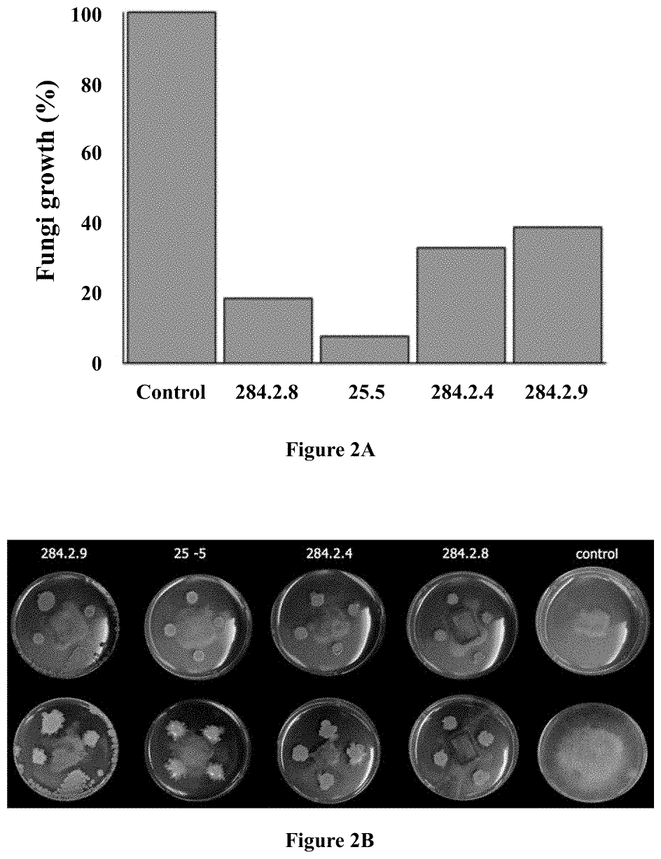

The antifungal activity of the bacteria described in Table 2, was also tested against Botrytis cinereal. Four isolates, 25.5, 284.2.8, 284.2.4, and 284.2.9, showed a significant antifungal growth with growth inhibition of 92.5%, 88.7%, 88.4%, and 81.6%, respectively (FIG. 2).

### Example 2

### Isolates 25.5 and 284.2.9 Inhibit Botrytis cinerea in Leaves

The biocontrol activity of the isolates against Botrytis cinerea was further tested in leaves. Isolates, 25.5, 284.2.4, 284.2.8, and 284.2.9, were examined in a pepper leaves experiment, as follows: pepper detached leaves (Capsicum annuum) were placed upside down in plastic containers (30×45×15 cm) with a grid while a wet filter paper was placed beneath the grid to maintain high humidity. Botrytis cinerea was grown in the 0.25 potato dextrose broth (PDB) for five days at 25° C. under shaking at 150 rpm. 20 μl of grown Botrytis cinerea spore suspension were placed on the lower surface of the leaves. The leaves were left at room temperature for 30 minutes to allow the spores to settle down; the boxes were then closed with a plastic transparent cover in order to maintain air humidity above 95% and incubated at 20° C. with a 12 hour photoperiod in the growth room. Then fungal growth and disease evaluation were measured by measuring the radius of the gray mold lesion growth. Bacterial isolates were placed on the leaves before, after or simultaneously with the fungus application.

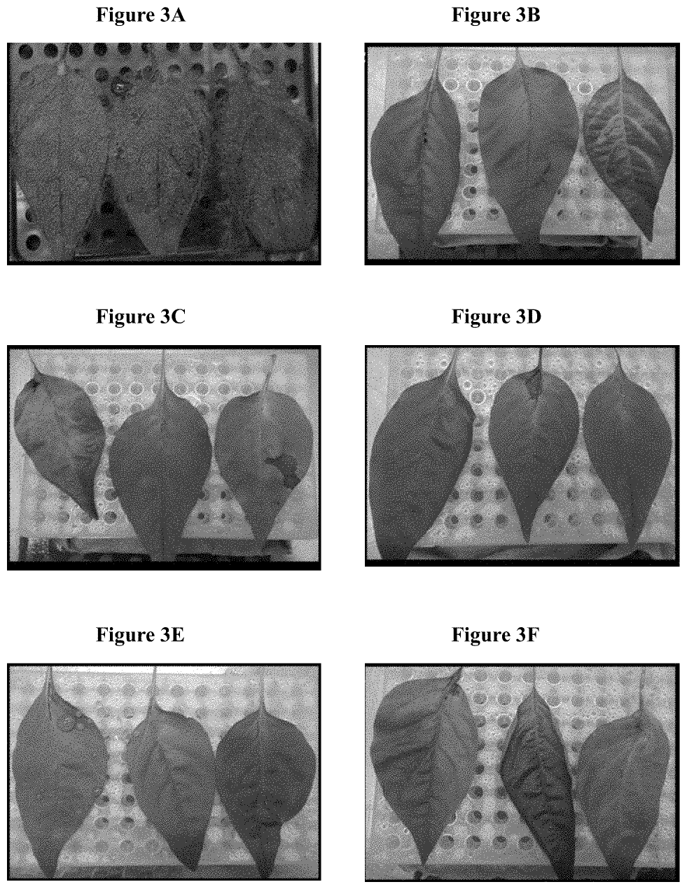

Out of the tested isolates, isolates 25.5, and 284.2.9 showed the most significant disease inhibition compared to a control (containing no bacterial isolates) (FIG. 3).

### Example 3

### Isolates 25.5 and 284.2.9 Inhibit Botrytis cinerea in Whole Plants

Isolates 25.5, and 284.2.9, that exhibited the most significant inhibition of Botrytis cinerea disease in leaf experiment, were tested in whole pepper plants. The pepper plants were wounded by decapitation, as follows: Botrytis spore suspensions were applied to the wounds as one drop of 20 μl and drying time was allowed. Then different bacteria isolates were applied one day before or one day after fungi inoculation, as one or two drops from a glass pipet. Plants were covered with transparent plastic sacks and inoculated for 10-14 days at 20° C. with a 12 hour photoperiod in the growth room for three weeks.

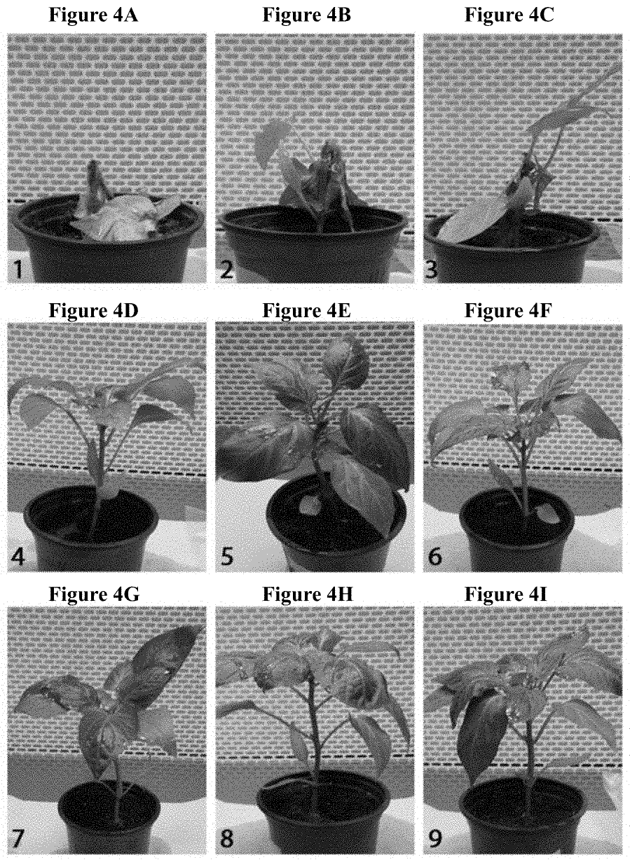

Under in vitro conditions, in temperature-controlled growth chamber, disease appeared within 7 days of pathogen application. The disease initially appeared on leaves as spots and blight at the inoculation spot. The disease rapidly developed at the petiole region and moved along margins towards the center of the plant (FIG. 4).

Effective biocontrol by the 25.5 isolate, and the 284.2.9 isolate was observed in all treatments (FIG. 4).

### Example 4

### T209.2 Isolates Surpass a Commercial Formula in Botrytis cinereal Inhibition

The activity of T209.2 against Botrytis cinereal was compared to “Switch”, a commercial bio-based formulation. The experiment was performed in pepper leaves as previously described in example 2.

The isolate surpassed the activity of commercial formulation, displaying better control of Botrytis cinerea infection in affected leaves (FIG. 5).

### Example 5

### Bacterial Sequencing and Identification

The isolates that were found to have fungicidal activity were further subjected to PCR protocol using long 16S rDNA primer set (Table 3). Briefly; 25 μl PCR reaction contained a 5 μl (of 1:100 diluted stock bacterial isolates in −80° C. with BPS following 3 cycles of freeze and thaw); 1 μl of mixed primer [100 pmol], 0.4 μl of DreamTaq DNA polymerase (Massachusetts, USA); 2.5 μl DreamTaq buffer; 2.5 μl BSA (10 mg/ml); 2 μl dNTPs; and 11.6 μl DNA/RNA free molecular water (sigma, Israel). Then, PCR reactions were placed in the thermocycler according to the following protocol; 90 s at 95° C.; 30 cycles of: 30 s at 94° C.; 30 s at 60° C.; 120 s at 72° C., and a final extension of 10 min at 72° C. Following PCR, the product was cleaned by cutting the amplified band from 1.5% agarose gel and purified using GenElute Minus EtBr Spin Column following the manufacturer protocol (sigma, Israel) and send to MCLAB for sequencing from both ends (California, USA).

TABLE 3
16S rDNA amplification primers.

| Primer | Sequence |
|---|---|
| Forward (SEQ ID NO: 4) | 5′-AGAGTTTGATMCTGGCTCAG-3′ |
| Reverse (SEQ ID NO: 5) | 5′-TACGGYTACCTTGTTACGACTT-3′ |

The M position in SEQ ID NO: 4, can be an A or C nucleotide. The Y position in SEQ ID NO: 5, can be an C or T nucleotide.

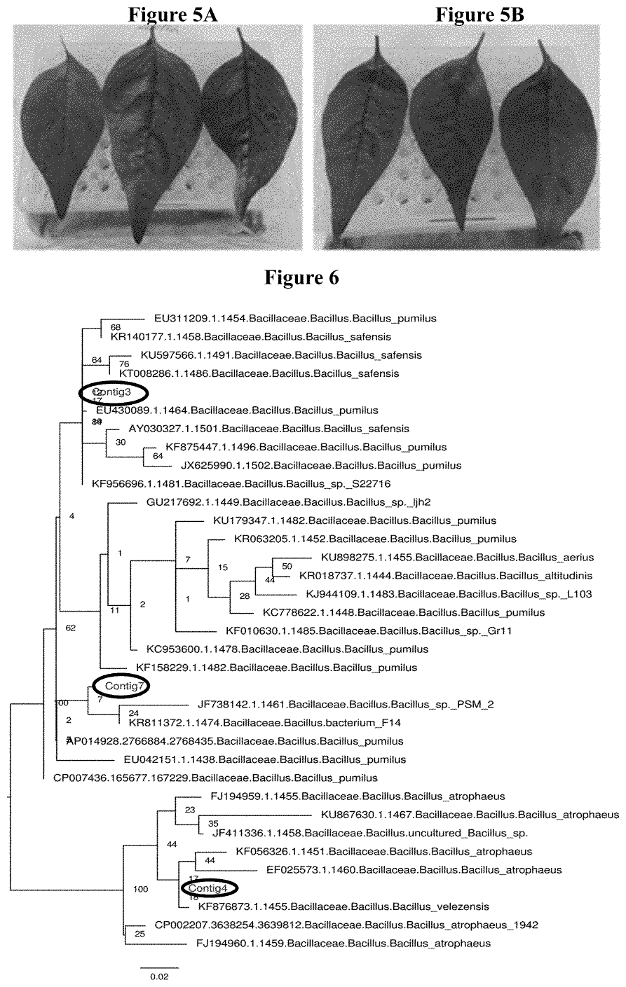

All obtained 16S rDNA sequences were checked for quality control, then contigs were constructed from both reads of forward and reverse primers using ChromasPro V2.1.8, and all low quality and incomplete sequences were removed. The inventors have also trimmed the first 25 bases and truncated the length of the sequences to 750 bp, due to low quality bp at the end of the sequences. Afterwards, sequences were blasted against silva database V128 using blastn software. Then sequences were clustered using VSEARCH software with 99.9% similarity. Upon clustering, all sequences were aligned using MAFFT sequence aligner. Then gaps with less than 20% of sequences were removed using trimAL. Then phylogenetic trees were generated using RAxML tool. Similar contigs were checked in the phylogeny tree and also checked for their alignment and Chromas. Isolates showing the same sequences were joint together (Table 4) and conclusive phylogenetic trees were represented (FIGS. 6-8).

TABLE 4
Contig numbers for isolates showing antifungal properties

| Contigs | Isolate name | SEQ ID NO. |
|---|---|---|
| Contig 5 | 25.2, 25.5, 284.2.8, 209.1, 23.3, | SEQ ID NO: 3 |
| | 284.2.9 | |
| Contig 10 | 284.2.4, 284.2.5 | SEQ ID NO: 2 |
| Contig 7 | 23.1 | SEQ ID NO: 8 |
| Contig 1 | 25.1 | SEQ ID NO: 1 |
| Contig 3 | 25.3 | SEQ ID NO: 6 |
| Contig 4 | 209.2 | SEQ ID NO: 7 |

Contigs 3, 4 and 7 were found to be identical to Bacillus pumilus and Bacillus atrophaeus (FIG. 6).

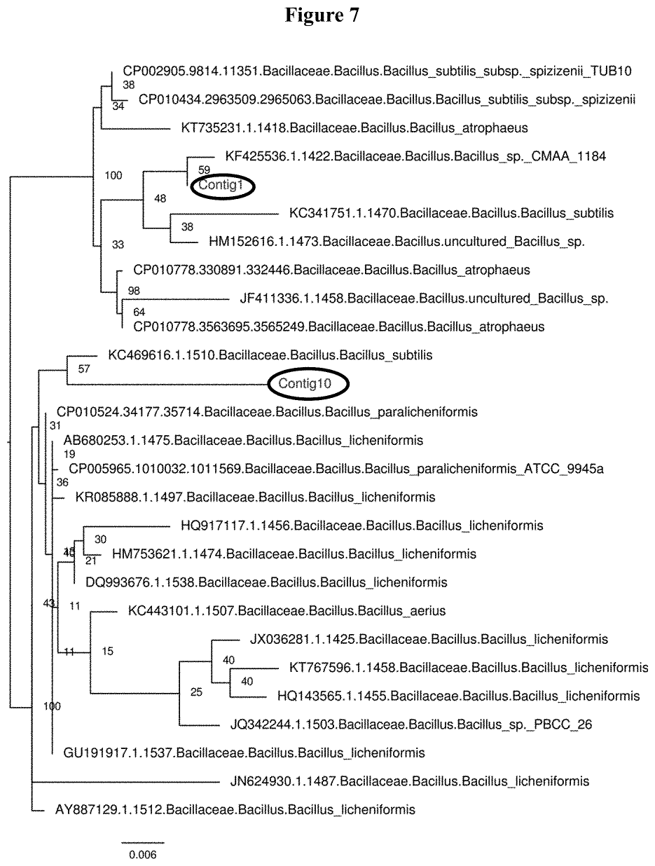

Contig 1 was found to have 99.52% sequence similarity to its closest related sequence, having the accession number KT735231, belonging to Bacillus atrophaeus, with several substitutions at positions 104, 850, 1189, 1194, 1284 and 1558. Contig 10 was found to have 98.07% sequence similarity, with numerous substitutions, to its closest related sequence having the accession number KC469616, belonging to Bacillus subtilis. Thus, both Contig 1 and Contig 10 were identified as new isolated strains (FIG. 7).

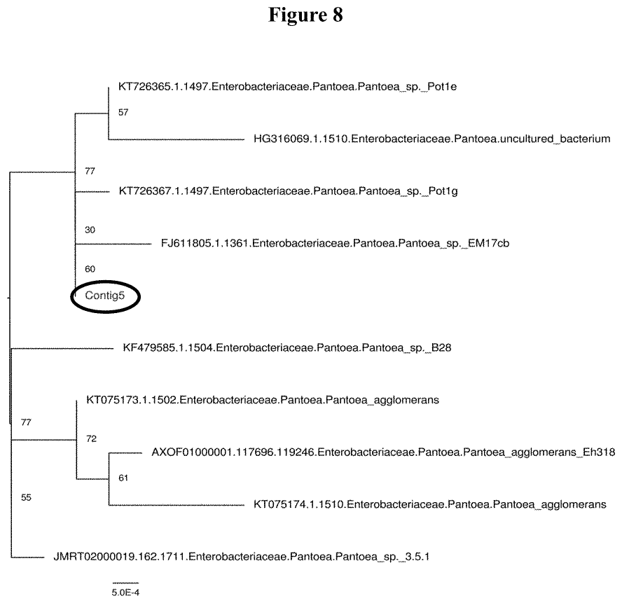

Additionally, Contig 5 was identified as a new isolate, closely related to Entrobacteriacecea Pantoea with sequence similarity of 99.81%. Contig 5 comprised most of the tested isolates and also showed the most significant results. The NCBI database showed Contig 5 is closely related to sequences having the accession numbers KT726367 and KT726365. Other closely related sequences to Contig 5 were found to belong to uncultured Pantoea spp_EM17cb, a non-pathogenic strain (FIG. 8). Contig 5 was found to have substitutions at positions 63 and 97 compared to the KT726365 and KT726367 accession numbers, respectively, and a ubiquitous substitution of N (any nucleotide) with adenine at positions 1,011 and 1,013.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1-4. (canceled)

5. A composition comprising bacteria, wherein at least 90% of said bacteria are bacteria comprising a polynucleotide molecule comprising SEQ ID NO: 1, bacteria comprising a polynucleotide molecule comprising SEQ ID NO: 2, or both, and an artificial carrier or diluent.

6. The composition of claim 5, further comprising bacteria comprising a polynucleotide molecule comprising SEQ ID NO: 3.

7. The composition of claim 6, wherein said bacteria comprising a polynucleotide molecule comprising SEQ ID NO: 3, belongs to Pantoea.

8. The composition of claim 5, wherein said polynucleotide sequence comprising any one of SEQ ID Nos: 1-3 is a 16S-rDNA sequence.

9. A method for inhibiting a fungus growth, comprising contacting a surface susceptible to fungus growth or fungus with any one of:
(a) isolated bacteria comprising a polynucleotide molecule comprising SEQ ID NO: 1;
(b) isolated bacteria comprising a polynucleotide molecule comprising SEQ ID NO: 2;
(c) isolated bacteria comprising a polynucleotide molecule comprising SEQ ID NO: 3;
(d) any combination of (a), (b), and (c); and
(e) an effective amount of the composition of claim 5, thereby inhibiting the fungus.

10. The method of claim 9, wherein said fungus is selected from the group consisting of: Pythium, Botrytis, Rhizoctonia, and Fusarium.

11. The method of claim 9, wherein said surface susceptible to fungus growth is a plant surface.

12. The composition of claim 5, wherein said bacteria comprising a polynucleotide molecule comprising SEQ ID NO: 1 or SEQ ID NO: 2, belongs to Bacillus.

13. The composition of claim 5, having a fungicidal activity
